(19) United States
(12) Patent Application Publication
(10) Pub. No.: US 2014/0334598 A1
(43) Pub. Date: Nov. 13, 2014

(54) X-RAY IMAGING APPARATUS AND METHOD OF CONTROLLING THE SAME

(71) Applicant: SAMSUNG ELECTRONICS CO., LTD., Suwon-si (KR)

(72) Inventor: Si Won PARK, Suwon-si (KR)

(73) Assignee: SAMSUNG ELECTRONICS CO., LTD., Suwon-si (KR)

(21) Appl. No.: 14/271,911

(22) Filed: May 7, 2014

(30) Foreign Application Priority Data
May 7, 2013 (KR) 10-2013-0051342

(51) Int. Cl. A61B 6/00

(52) U.S. Cl. CPC A61B 6/502
USPC 378/37

(57) ABSTRACT

An X-ray imaging apparatus may include a frame, an X-ray generation unit that radiates X-rays onto an object, an X-ray detection unit that detects the X-rays transmitted by the object, and a compression paddle that is combined with the frame so that the compression paddle is placed above the X-ray detection unit and compresses the object. A frame combining unit may form at least one surface of the compression paddle and may be combined with the frame. A plurality of protrusions may be provided at the frame combining unit so that a type of the compression paddle may be recognized, thereby preventing an error in recognizing the type of the compression paddle. Also, the compression paddle may be combined with the frame by using the protrusions to stably combine the compression paddle with the frame.

### CROSS-REFERENCE TO RELATED APPLICATIONS

This application claims the benefit of Korean Patent Application No. 10-2013-0051342, filed on May 7, 2013 in the Korean Intellectual Property Office, the disclosure of which is incorporated herein by reference.

### BACKGROUND

1. Field

Embodiments disclosed herein relate to an X-ray imaging apparatus in which an X-ray image is generated by transmitting X-rays by an object, and a method of controlling the same.

2. Description of the Related Art

X-ray imaging apparatuses generally refer to and include apparatuses that are capable of obtaining an image inside an object by radiating X-rays onto the object and using the X-rays transmitted by (reflected from) the object. Since transmittance of X-rays varies according to the characteristics of a material used to form the object, an internal structure of the object can be imaged by detecting an intensity or strength of the X-rays transmitted by the object.

A process in which a breast is used as an object, using an X-ray imaging apparatus, is generally referred to as mammography. Since the breast is a body part in which a breast tissue and a fat tissue are developed, X-ray photographing should be performed in a state in which the breast placed between an X-ray generation unit and an X-ray detection unit is compressed using a compression paddle, so that an X-ray image that clearly represents an internal structure of the breast can be obtained.

Several types of compression paddles may be used according to the type or characteristics of the breast which corresponds to the object which is radiated. The compression paddle should be combined with a frame of the X-ray imaging apparatus so that a controller can recognize the type of the compression paddle. However, an error occurs in this procedure.

### SUMMARY

Therefore, it is an aspect of the example embodiments disclosed herein to provide an X-ray imaging apparatus having an improved structure of a frame combining unit in which a compression paddle is combined with a frame, so that the type of the compression paddle can be recognized, and a method of controlling the X-ray imaging apparatus.

Additional aspects of the example embodiments disclosed herein will be set forth in part in the description which follows and, in part, will be apparent from the description, or may be learned by practice of the invention.

In accordance with an aspect of the example embodiments disclosed herein, an X-ray imaging apparatus may include a frame, an X-ray generation unit that is placed at one side of the frame, and which generates X-rays, and radiates the X-rays onto an object, an X-ray detection unit that is placed at the other side of the frame, and which detects the X-rays transmitted by the object, and converts the detected X-rays into electrical signals. The X-ray imaging apparatus may further include a compression paddle that is combined with the frame so that the compression paddle is placed above the X-ray detection unit and compresses the object, a frame combining unit that forms or comprises at least one surface of the compression paddle and is combined with the frame, and a plurality of protrusions provided at the frame combining unit so that a type of the compression paddle is capable of being recognized through or based upon the plurality of protrusions.

The X-ray imaging apparatus may further include a compression paddle combining unit which is disposed on the frame and with which the frame combining unit is combined.

The compression paddle combining unit may include a plurality of grooves having a shape corresponding to the protrusions so that the protrusions are accommodated in the plurality of grooves.

The X-ray imaging apparatus may further include a transmitting unit that generates signals so that insertion of the protrusions is capable of being recognized. For example, the transmitting unit may be disposed at an inner side of the plurality of grooves.

The compression paddle may be disposed to move upward and downward relative to the frame.

The frame combining unit may be disposed to be inserted into an inner side of the frame and to contact the compression paddle combining unit.

The frame combining unit may include a neck part inserted into the inner side of the frame, and the plurality of protrusions may be provided on at least one surface of the neck part.

The frame combining unit may include an insertion part that protrudes from both sides of the frame combining unit so that the insertion part is capable of sliding and being inserted into the compression paddle combining unit, and the compression paddle combining unit may include an accommodation part having a shape corresponding to the insertion part.

A plurality of protrusions may be placed on at least one surface of the frame combining unit which is combined with the compression paddle combining unit.

The plurality of protrusions may include a first state in which the protrusions protrude to be inserted into the grooves disposed in at least one surface of the compression paddle combining unit and a second state in which the protrusions are accommodated in the inner side of the frame combining unit.

The X-ray imaging apparatus may further include a controller that recognizes the type of the compression paddle through the plurality of protrusions and controls an X-ray radiation region of the X-ray generation unit.

The X-ray generation unit may include an X-ray source that generates X-rays and a collimator that adjusts a radiation region of the X-rays radiated from the X-ray source, and the controller may control the X-ray radiation region of the collimator according to the type of the compression paddle.

In accordance with another aspect of the example embodiments disclosed herein, a method of controlling an X-ray imaging apparatus including a frame, an X-ray generation unit that generates X-rays and radiates the X-rays onto an object, an X-ray detection unit that detects the X-rays transmitted by the object, and a compression paddle that compresses the object, includes: combining the compression paddle with the frame, recognizing a type of the compression paddle using a plurality of protrusions provided on at least one surface of the compression paddle; and controlling the X-ray generation unit based on the recognized type of the compression paddle.

The recognizing of the type of the compression paddle may include recognizing whether the protrusions of the frame combining unit are combined with grooves of a compression paddle combining unit with which the compression paddle of the frame is combined.

A transmitting unit placed at the grooves may transmit data regarding the compression paddle.

The combining of the compression paddle may include combining the compression paddle with the frame so that the protrusions protrude.

The combining of the compression paddle may further include placing the object on the X-ray detection unit and then combining the compression paddle with the frame.

The combining of the compression paddle may further include combining the compression paddle with the frame before placing the object on the X-ray detection unit.

The controlling of the X-ray generation unit may include determining a first object region according to the type of the compression paddle and determining a second object region according to the object placed on the X-ray detection unit.

The controlling of the X-ray generation unit may include controlling a collimator and adjusting a radiation region of the X-rays.

The controlling of the collimator may include determining an object region according to a size of the object by recognizing the type of the compression paddle combined with the frame and setting a radiation region of the X-rays to correspond to the object region.

The controlling of the collimator may further include controlling the collimator so that the X-rays generated in the X-ray source are capable of being radiated into the set radiation region.

In accordance with another aspect of the example embodiments disclosed herein, an X-ray imaging apparatus may include a frame, a compression paddle to compress an object disposed between the compression paddle and an X-ray detection unit, a frame combining unit connected to the compression paddle, which includes a plurality of protrusions disposed on at least one surface of the frame combining unit, and a compression paddle combining unit connected to the frame and to the frame combining unit, which includes a plurality of grooves disposed on at least one surface of the compression paddle combining unit to accommodate the plurality of protrusions.

The X-ray imaging apparatus may further include a controller to recognize a type of the compression paddle based upon a determination as to a number of protrusions which are inserted into the plurality of grooves.

The compression paddle combining unit may further include a transmitting unit to transmit a signal to the controller indicating whether a first protrusion of the frame combining unit is inserted into a first groove of the compression paddle combining unit.

The transmitting unit may include a transmitting body, a connection part disposed on the transmitting body, a first terminal to receive a force when the first protrusion is inserted into the first groove, and a metal plate connected to the first terminal on a first end and connected to the transmitting body on a second end, to contact the connection part when the first terminal receives the force. The transmitting unit may transmit the signal to the controller when the connection part is contacted by the metal plate. The compression paddle may include a compression surface to compress the object, and a first compression surface frame and a second compression surface frame adapted to accommodate the compression surface. The frame combining unit may be integrally formed together with the first compression surface frame and the second compression surface frame.

### BRIEF DESCRIPTION OF THE DRAWINGS

These and/or other aspects of the example embodiments disclosed herein will become apparent and more readily appreciated from the following description of the embodiments, taken in conjunction with the accompanying drawings of which:

FIG. 1 illustrates an exterior of an X-ray imaging apparatus in accordance with an example embodiment;

FIG. 2 is an enlarged view of a compression paddle of the X-ray imaging apparatus illustrated in FIG. 1;

FIG. 3 illustrates a frame combining unit of the compression paddle of the X-ray imaging apparatus of FIG. 1;

FIG. 4 illustrates a compression paddle combining unit of a frame of the X-ray imaging apparatus of FIG. 1;

FIG. 5 illustrates a transmitting unit of the compression paddle combining unit of the X-ray imaging apparatus of FIG. 1;

FIG. 6 is a side view illustrating a side surface of a compression paddle of an X-ray imaging apparatus in accordance with another aspect of the example embodiments;

FIG. 7A illustrates a state in which protrusions of a frame combining unit of a compression paddle of the X-ray imaging apparatus illustrated in FIG. 6 protrude;

FIG. 7B illustrates a state in which the protrusions of the frame combining unit of the compression paddle of the X-ray imaging apparatus of FIG. 6 are accommodated;

FIG. 8 illustrates a compression paddle combining unit of a frame of the X-ray imaging apparatus of FIG. 6;

FIG. 9 illustrates a compression paddle of an X-ray imaging apparatus in accordance with another embodiment of the example embodiments;

FIG. 10 illustrates a frame combining unit of the compression paddle of the X-ray imaging apparatus illustrated in FIG. 9;

FIG. 11 is a control block diagram of the X-ray imaging apparatus illustrated in FIG. 1; and

FIG. 12 is a flowchart illustrating a method of controlling an X-ray imaging apparatus, in accordance with an example embodiment.

### DETAILED DESCRIPTION

Reference will now be made in detail to the example embodiments disclosed herein, examples of which are illustrated in the accompanying drawings, wherein like reference numerals refer to like elements throughout.

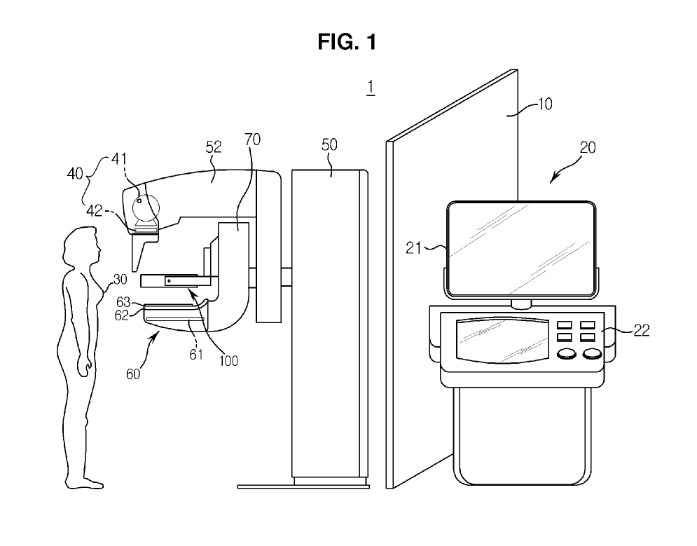

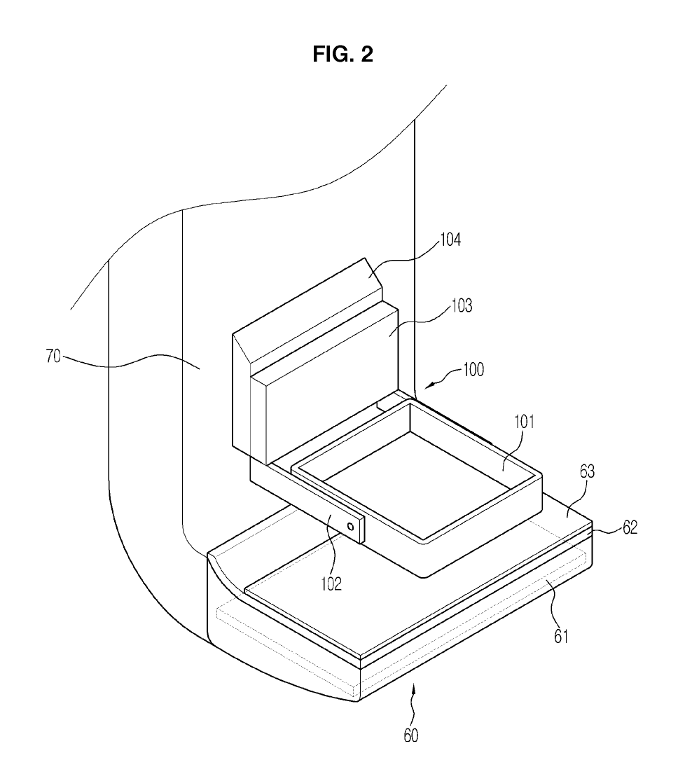

FIG. 1 illustrates an exterior of an X-ray imaging apparatus in accordance with an example embodiment, and FIG. 2 is an enlarged view of a compression paddle of the X-ray imaging apparatus illustrated in FIG. 1.

The X-ray imaging apparatus in accordance with an example embodiment may capture an image of an object (for example, a breast). Due to characteristics of the breast in which a breast tissue and a fat tissue are developed, the X-ray imaging apparatus for capturing the image of the breast has a configuration in which X-rays may be radiated in a state in which the breast is compressed.

As illustrated in FIGS. 1 and 2, an X-ray imaging apparatus 1 in accordance with an example embodiment may include an X-ray generation unit 40 that generates X-rays and radiates the X-rays onto an object 30, an X-ray detection unit 60 that detects the X-rays transmitted (or reflected) by the object 30, and a compression paddle 100 that is placed above the X-ray detection unit 60 and compresses the object 30. That is, the object may be placed between compression paddle 100 and the X-ray detection unit 60. The X-ray generation unit 40 and the X-ray detection unit 60 may be connected to a frame 70 via a connection arm, and the frame 70 may support the X-ray generation unit 40 and the X-ray detection unit 60. For example, the X-ray generation unit 40 may be connected to the frame 70 via a connection arm 52, as shown in FIG. 1. The frame 70 may be mounted on a gantry 50. A high-voltage generation device may be disposed inside the gantry 50 and may supply a high voltage to the X-ray generation unit 40.

The X-ray generation unit 40 may include an X-ray source 41 that generates X-rays and a collimator 42 that adjusts a radiation region of the X-rays in which the X-rays are radiated from the X-ray source 41.

When the object 30 is a breast, the breast needs to be compressed in a direction perpendicular to the ground and a thickness of the breast needs to be decreased so as to obtain a clearer and more accurate image. Thus, the breast is placed between the compression paddle 100 and the X-ray detection unit 60, and the X-rays are radiated in a state in which the object 30 is compressed by the compression paddle 100. The compression paddle 100 may move upward and downward. To this end, the compression paddle 100 may include a compression paddle adjustment lever (not shown).

The compression paddle 100 may include a compression surface 101 that compresses the breast that corresponds to the object 30, and compression surface frames 102 and 103 that are placed around the compression surface 101. The compression surface 101 may be placed between the X-ray generation unit 40 and the X-ray detection unit 60. Thus, the X-rays may be transmitted by the compression surface 101 and may be transmitted by the breast that corresponds to the object 30. The compression surface 101 may be formed of a transparent material not to affect the result of inspection.

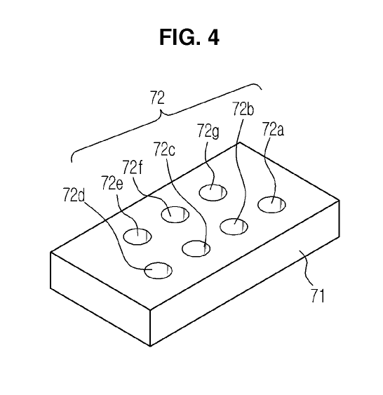

The compression frames 102 and 103 may extend to a frame combining unit 104 combined with the frame 70. The frame combining unit 104 may be separately combined with the compression paddle 100 and may be provided integrally with the compression surface frames 102 and 103. For example, the compression surface frames 102 and 103 may include a first part 102 combined with the compression surface 101 and a second part 103 combined with the frame combining unit 104. However, the example embodiments of disclosed herein are not limited thereto. At least a part of the frame 70 may include a compression paddle combining unit 71 (for example, as shown in FIG. 4) with which the compression paddle 100 may be combined. The frame combining unit 104 and the compression paddle combining unit 71 will be described below.

The X-ray detection unit 60 may include an X-ray detector 61 that detects the X-rays transmitted by the object 30, converts the X-rays into electrical signals, and obtains X-ray data from the electrical signals. A sheet 62 on which the breast that is the object 30 may be put, may be disposed in an upper portion of a housing in which the X-ray detector 61 is accommodated. The sheet 62 may be implemented with a material and color that minimize the effect on X-ray transmission. For example, a carbon sheet may be used as the sheet 62.

The X-ray detector 61 may be classified according to a material composition method, a method of converting detected X-rays into electrical signals, and a method of obtaining X-ray data.

For example, the X-ray detector 61 may be classified as a single type device or a combined type device according to the material composition method.

When the X-ray detector 61 is configured as the single type device, a part for detecting X-rays and generating electrical signals and a part for reading and processing the electrical signals may be formed as a semiconductor of a single material or may be manufactured in a single process, such as when a charge coupled device (CCD) that is a light receiving device or a complementary metal oxide semiconductor (CMOS) is used as the X-ray detector 61.

When the X-ray detector 61 is configured as the combined type device, a part for detecting X-rays and generating electrical signals and a part for reading and processing the electrical signals may be formed of different materials or may be manufactured in different processes, such as when X-rays are detected using a light receiving device. The light receiving device may include, for example, a photodiode, a CCD, or cadmium zinc telluride (CdZnTe) and the electrical signals may be read and processed using a complementary metal oxide semiconductor read out integrated circuit (CMOS ROIC), when the X-rays are detected using a strip detection unit and the electrical signals are read and processed using the CMOS ROIC, and when an a-Si or a-Se flat panel system is used as the X-ray detector 61.

The X-ray detector 61 may be classified as a direct conversion type or an indirect conversion type according to a method of converting X-rays into electrical signals.

In the direct conversion method, a pair of electrons and holes that are temporarily generated in a light receiving device due to radiated X-rays and that are moved to an anode and a cathode due to an electric field applied to both ends of the light receiving device, are converted into electrical signals. In the direct conversion method, a-Se, CdZnTe, HgI₂, PbI₂, or the like may be used as the light receiving device; however, the example embodiments disclosed herein are not limited thereto.

In the indirect conversion method, a scintillator may be provided between a light receiving device and an X-ray generation unit. The light receiving device may detect photons having a wavelength in a visible light region as X-rays radiated by the X-ray generation unit react with the scintillator, and the detected photons may be converted into electrical signals. In the indirect conversion method, a-Si may be used as the light receiving device; however, the example embodiments disclosed herein are not limited thereto. Also, a gadolinium oxysulfide (GADOX) scintillator having a thin film shape or a micro pillar-shaped or needle-shaped CSI (TI) scintillator may be used; however, the example embodiments disclosed herein are not limited thereto.

Also, the X-ray detector 61 may be classified according to a method of obtaining X-ray data as a charge integration mode in which charges are stored for a predetermined amount of time and then signals are obtained from the charges or a photon counting mode in which photons having a threshold energy or more are counted whenever a signal is generated from single X-ray photons.

Any one or more of the above-described methods, such as the material composition method, the method of converting detected X-rays into electrical signals, and the method of obtaining X-ray data, may be applied to the X-ray detector 61 used in the example embodiments disclosed herein.

X-ray data obtained by the X-ray detector 61 may be transmitted to a host device 20. The host device 20 may include a display 21 on which an X-ray image is displayed, and an input unit 22 that inputs an instruction regarding an operation of the X-ray imaging apparatus 1. The host device 20 may be used to communicate with and control functions of, the X-ray generation unit 40, the X-ray detection unit 60, and the compression paddle 100, for example. The host device 20 may communicate and control the X-ray generation unit 40, the X-ray detection unit 60, and the compression paddle 100, for example, over a wired or wireless network, or a combination thereof.

The display 21 may include a liquid crystal display (LCD), a light emitting diode (LED) display, an organic light emitting diode (OLED) display, a plasma display panel (PDP), a cathode ray tube (CRT) display, and the like, for example. However, the disclosure is not so limited and may include other types of displays.

The input unit 22 may include, for example, one or more of a keyboard, a mouse, a joystick, a button, a switch, an electronic pen or stylus, a device to recognize a gesture (e.g., movement of a body part), an input sound device (e.g., a microphone to receive a voice command), an output sound device (e.g., a speaker), a track ball, a remote controller, a portable (e.g., a cellular or smart) phone, a tablet PC, a pedal or footswitch, a virtual-reality device, and so on. The input unit may further include a haptic device to provide haptic feedback to a user. The input unit may also include a touchscreen, for example. One or more devices may be utilized to input an instruction to control an operation of the X-ray imaging apparatus 1.

The X-ray detection unit 60 may further include a touch sensor 63 that detects a touch with the object 30. The touch sensor 63 may be placed at an upper side of the X-ray detector 61. According to an example embodiment, the touch sensor 63 may be mounted on the sheet 62. Thus, the breast that is the object 30 is placed on the touch sensor 63 so as to perform X-ray photographing, and a user (for example, a radiology technician), may adjust the compression paddle adjustment lever (not shown) so that the compression paddle 100 may compress the breast to a predetermined thickness.

Meanwhile, a shield 10 that partitions off a region in which X-ray photographing is performed on a patient and a region in which the radiology technician manipulates the host device 20 may be installed so as to prevent unnecessary exposure to radiation. The shield 10 may be formed of a material, such as lead (Pb), that absorbs X-rays. For example, the radiology technician may compress the breast, check a region in which the compressed breast is placed (for example, with his/her eyes), move to the host device 20 placed opposite to the shield 10, and adjust the collimator 42 (e.g., via the input unit), to thereby radiate X-rays and capture an image of the breast. However, this is just one example, and other scenarios or sequences of operations are possible to capture the image of the breast or of any object generally.

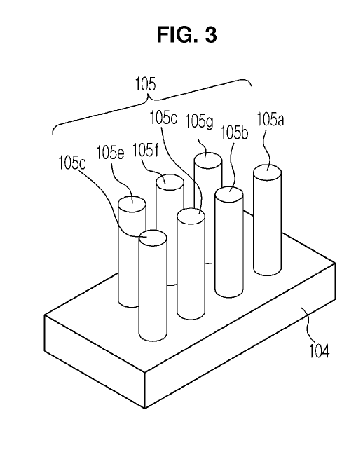

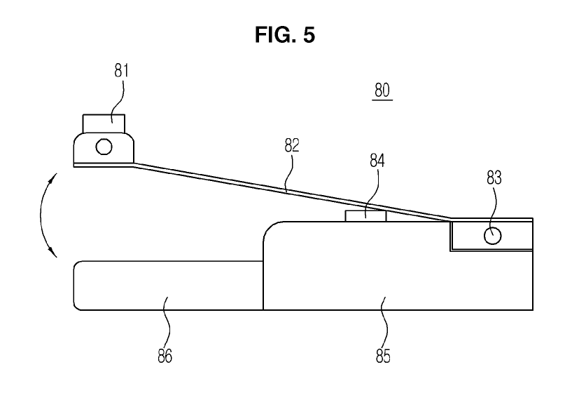

FIG. 3 illustrates a frame combining unit of the compression paddle of the X-ray imaging apparatus of FIG. 1, FIG. 4 illustrates a compression paddle combining unit of a frame of the X-ray imaging apparatus of FIG. 1, and FIG. 5 illustrates a transmitting unit of the compression paddle combining unit of the X-ray imaging apparatus of FIG. 1.

As illustrated in FIGS. 3 through 5, the frame combining unit 104 may include a plurality of protrusions 105 according to the type of the compression paddle 100. Although seven protrusions (protrusions 105a through 105g) are shown in the drawings, the example embodiments disclosed herein are not limited thereto. For example, the frame combining unit 104 may include less than or more than seven protrusions. If one protrusion among the seven protrusions 105 is defined as a first protrusion 105a, two compression paddles may be defined as when the frame combining unit 104 includes the first protrusion 105a and when the frame combining unit 104 does not include the first protrusion 105a. In this way, types of several compression paddles 100 may be defined depending on whether the frame combining unit 104 includes at least one of the plurality of protrusions 105. That is, as illustrated in the drawings, when seven protrusions 105 are placed on the frame combining unit 104, the total number of types of compression paddles may correspond to 2⁷, i.e., 128 compression paddles can be defined. By way of another example, when six protrusions 105 are placed on the frame combining unit 104, the total number of types of compression paddles may correspond to 2⁶, i.e., 64 compression paddles can be defined.

For example, as shown in FIG. 4, the frame 70 may include the compression paddle combining unit 71 with which the frame combining unit 104 of the compression paddle 100 may be combined. The compression paddle combining unit 71 may include a plurality of grooves 72 in which the plurality of protrusions 105 of the frame combining unit 104 may be accommodated. The plurality of grooves 72 (e.g., grooves 72a through 72g) may have a shape corresponding to the protrusions 105. A transmitting unit 80 that may generate signals so that insertion of the protrusions 105 can be recognized, may be disposed at an inner side of the grooves 72. That is, the transmitting unit 80 may be disposed in the form of a switch, as illustrated in FIG. 5; however, the embodiments disclosed herein are not limited thereto.

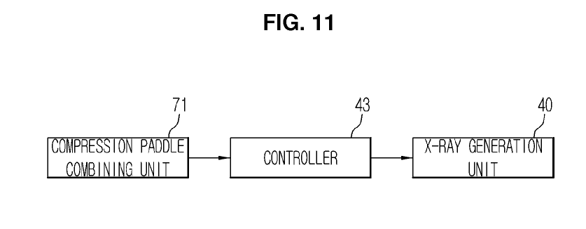

The protrusions 105 may be accommodated in the grooves 72. The protrusions 105 may be accommodated in the grooves 72 and may pressurize a terminal 81 of the transmitting unit 80 placed at the inner side of the grooves 72. The terminal 81 may be combined with a transmitting unit body 85 via a hinge 83 through a metal plate 82. As the terminal 81 is pressurized, the metal plate 82 may be moved in a direction of the transmitting unit body 85. Thus, a connection part 84 placed at the transmitting unit body 85 may be pressurized by the metal plate 82. That is, as shown in the example embodiment in FIG. 5, a directional force (e.g., vertical or horizontal, etc.) may be applied to the terminal 81 due to at least one protrusion 105 being accommodated in at least one groove 72. The directional force may cause the metal plate 82 to move inward such that a portion of the metal plate 82 causes an inward force upon connection part 84. Thus, the transmitting unit 80 may be turned on, and data representing that one or more of the protrusions 105 are accommodated in the grooves 72 may be transmitted to a controller 43 (see FIG. 11, for example). Also, when no protrusions 105 are accommodated in the grooves 72, a force for pressing the terminal 81 disappears. Thus, the metal plate 82 may be separated from the transmitting unit body 85, and no signals may be transmitted to the controller 43. By using the structure, signals representing that the protrusions 105 are accommodated in the grooves 72 may be transmitted to the controller 43, and the controller 43 may recognize the type of the compression paddle 100 by calculating the signals. That is, a terminal 81 may be separately provided for each protrusion and/or groove. For example, when a first protrusion is (or is not) accommodated in the respective groove corresponding to the first protrusion, the appropriate data may be obtained by a first terminal recognizing the presence or absence of the first protrusion in the first groove. A second terminal may be provided for a second protrusion and second groove, and so on. For example, the number of terminals may correspond to the number of grooves disposed in the compression paddle combining unit 71. The terminals may be connected to a common or separate transmitting unit body 85, and the data representing the state of the protrusions in the grooves may be separately transmitted or transmitted together to the controller 43. The transmitting unit 80 may further include an elastic member (not shown) so as to easily move the terminal 81.

In this way, due to the protrusions 105 placed at the frame combining unit 104, recognition of the type of the compression paddle 100 may be structurally performed. Thus, an error that occurs in recognizing the type of the compression paddle 100 may be reduced. Also, since the compression paddle 100 may be combined with the frame 70 using the protrusions 105, a combining force between the compression paddle 100 and the frame 70 may be increased so that the compression paddle 100 may be prevented from being separated from the frame 70 in an unintended state. That is, the stability of the compression paddle 100 and the frame 70 may be increased.

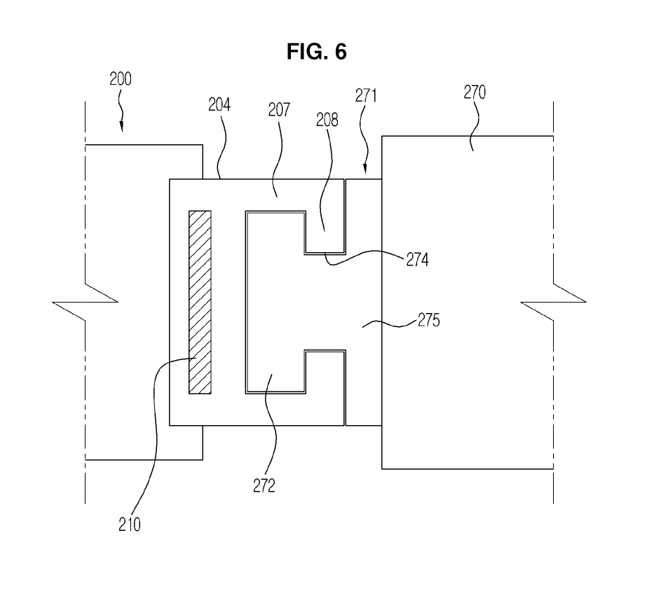

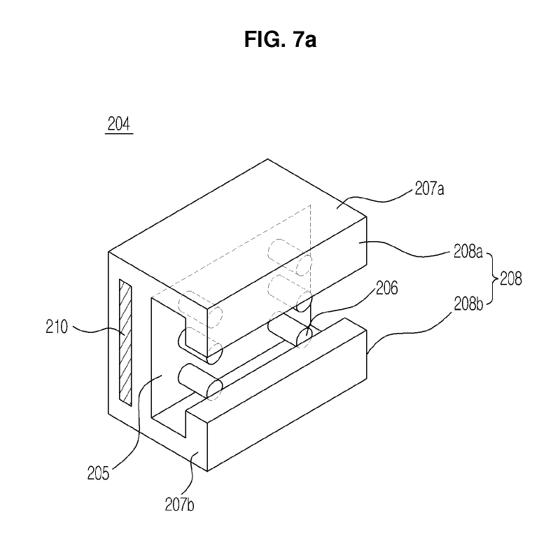

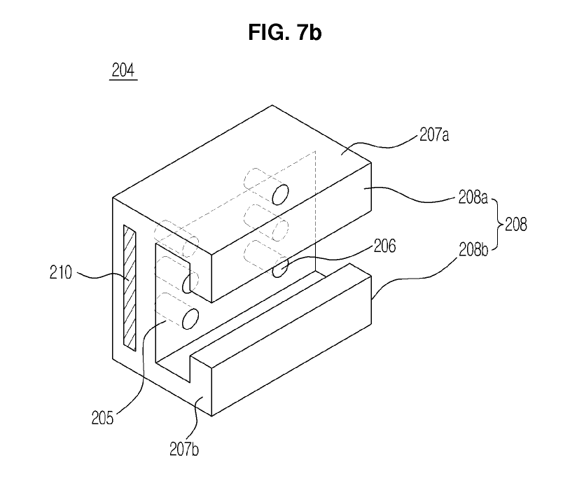

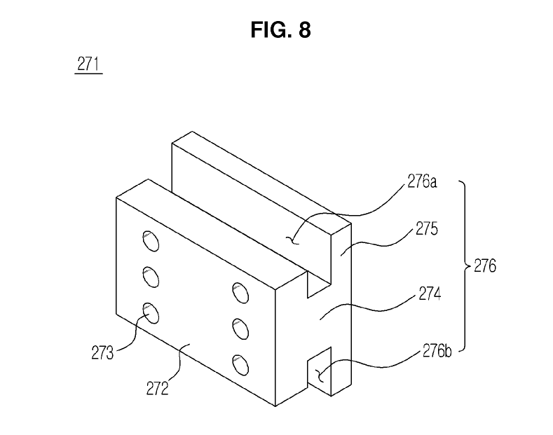

FIG. 6 is a side view illustrating a side surface of a compression paddle of an X-ray imaging apparatus in accordance with another aspect of the example embodiments disclosed herein. FIG. 7A illustrates a state in which protrusions of a frame combining unit of a compression paddle of the X-ray imaging apparatus illustrated in FIG. 6 protrude, FIG. 7B illustrates a state in which the protrusions of the frame combining unit of the compression paddle of the X-ray imaging apparatus of FIG. 6 are accommodated, and FIG. 8 illustrates a compression paddle combining unit of a frame of the X-ray imaging apparatus of FIG. 6.

As illustrated in FIGS. 6 through 8, a frame combining unit 204 of the X-ray imaging apparatus may slide toward a compression paddle combining unit 271 and may be inserted therein. To this end, the frame combining unit 204 may include an insertion part 207 that protrudes in a direction of a frame 270 from both sides of the frame combining unit 204 and that is inserted into the compression paddle combining unit 271. The insertion part 207 is disposed to extend to both sides of a frame combining unit body 205 and includes a first insertion part 207a and a second insertion part 207b.

The frame combining unit 204 may include an extension part 208 that extends from one side of the insertion part 207. The extension part 208 may be disposed to be bent in a direction perpendicular to the insertion part 207. The extension part 208 may be disposed at both sides of the frame combining unit body 205 and may include a first extension part 208a and a second extension part 208b. The first extension part 208a and the second extension part 208b may be disposed to face each other. For example, as can be seen from FIGS. 7A and 7B, the frame combining unit 204 may be formed to have a substantially C-shaped structure, and an interior portion which is hollow (except for the protrusions 206) may be substantially T-shaped.

The compression paddle combining unit 271 may include an accommodation part 276 for accommodating at least a part of the frame combining unit 204. The accommodation part 276 may have a shape corresponding to the insertion part 207 of the frame combining unit 204. In order to accommodate the extension part 208, a concave part 274 in which at least a part of the compression paddle combining unit body 275 is concavely inserted, may be provided. The insertion part 207 may be combined with the accommodation part 276, and the extension part 208 may be combined with the concave part 274. Since the insertion part 207 and the extension part 208 of the frame combining unit 204 are disposed at both sides of the frame combining unit body 205 (for example, upper and lower sides), the accommodation part 276 of the compression paddle combining unit 271 may also be disposed at both sides of the compression paddle combining unit body 275. That is, the accommodation part 276 may include a first accommodation part 276a and a second accommodation part 276b. For example, as can be seen from FIG. 8, the compression paddle combining unit 271 may be formed to have an asymmetrical or irregularly H-shaped structure, which may interlock, connect, or join together with the interior hollow portion of the frame combining unit 204. For example, the frame combining unit 204 may slide toward the compression paddle combining unit 271 and may be inserted therein. For example, the first extension part 208a and the second extension part 208b may slide and fit into the first accommodation part 276a and the second accommodation part 276b, respectively, while surface 272 may be pressed against or substantially adjacent to the inner side of the frame combining unit body 205 of the frame combining unit 204, so that the protrusions 206 protrude or are capable of protruding into the grooves 273.

A plurality of protrusions 206 may be used to recognize the type of compression paddle 200 and may be provided at at least a part of an inner side of the frame combining unit body 205. The plurality of protrusions 206 may define the type of the compression paddle 200 depending on whether the frame combining unit body 205 includes the protrusions 206, as described above. The plurality of protrusions may be arranged in a predetermined pattern, or may be randomly distributed on the inner side of the frame combining unit body 205. The protrusions may be spaced evenly from one another, or may be arranged at varying intervals. A plurality of grooves 273 into which the protrusions 206 may be inserted, may be placed or disposed in at least one surface of the compression paddle combining unit 271. For example, as shown in FIG. 8, the plurality of grooves 273 may be formed in a surface 272 that is closest to the frame combining unit 204. The grooves 273 may be provided to correspond to the shape and arrangement of the protrusions 206 which are formed on the inner side of the frame combining unit body 205. The transmitting unit 80 described above may be provided at an inner side of the grooves 273.

According to an example embodiment, the protrusions 206 may include a first state in which the protrusions 206 protrude to be inserted into the grooves 273 and a second state in which the protrusions 206 are accommodated in an inner side of the frame combining unit 204. FIG. 7A illustrates the first state, and FIG. 7B illustrates the second state. After the frame combining unit 204 is combined with the compression paddle combining unit 271 in the second state, the second state may be converted into the first state in which the protrusions 206 may be combined with the grooves 273. That is, the protrusions may be disposed within the inner side of the frame combining unit body 205 so that the frame combining unit 204 may be combined with the compression paddle combining unit 271. Then, after the frame combining unit 204 is combined with the compression paddle combining unit 271, the protrusions may be released from the inner side of the frame combining unit body 205 and protrude into the grooves (recesses) of the compression paddle combining unit 271. In an alternative embodiment, the protrusions may be composed of a flexible material which may bend to allow the frame combining unit 204 to be combined with the compression paddle combining unit 271.

A control unit 210 by which a user may adjust conversion of the protrusions 206 from the first state to the second state or from the second state to the first state may be provided at at least a part of the frame combining unit 204. For example, as shown in FIG. 6, the control unit 210 may be provided at a side surface of the frame combining unit 204; however, the example embodiments disclosed herein are not limited thereto. By way of example, if the user (or an external force) presses the control unit 210, the protrusions 206 may protrude. For example, the protrusions 206 may be held in the second state in which the protrusions 206 are disposed entirely, or substantially entirely, within the inner side of the frame combining unit body 205. When a user or external force presses the control unit 210, the protrusions 206 may emerge or be released from the inner side of the frame combining unit body 205 to protrude substantially outward from the inner side of the frame combining unit body 205. Additionally, the control unit 210 may be activated or triggered such that the protrusions 206 are withdrawn and the protrusions 206 are converted from the first state to the second state. Alternatively, the control unit 210 may be implemented through another method, for example, by pulling, or via a switch or lever. In an alternative embodiment, the number of protrusions in the frame combining unit which protrude from the frame combining unit may be adjustable. For example, a user may depress the control unit 210 a certain number of times to cause a certain number of protrusions to protrude or to be retracted.

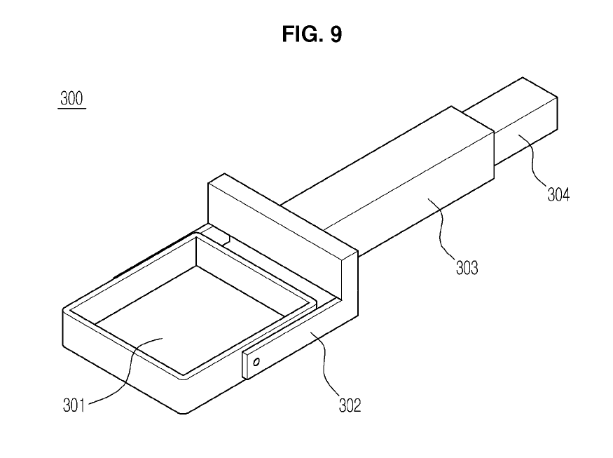

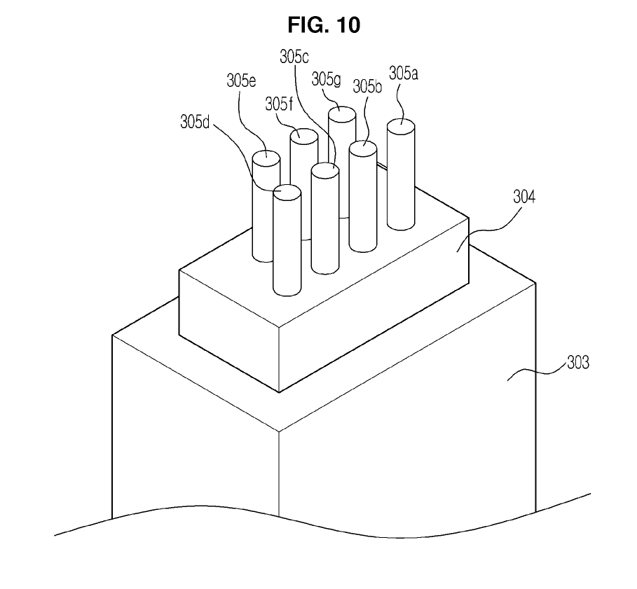

FIG. 9 illustrates a compression paddle of an X-ray imaging apparatus in accordance with an example embodiment, and FIG. 10 illustrates a frame combining unit of the compression paddle of the X-ray imaging apparatus illustrated in FIG. 9.

As illustrated in FIGS. 9 and 10, a frame combining unit 303 may be provided to be inserted into an inner side of a frame and to contact a compression paddle combining unit. That is, at least a part of the frame combining unit 303 may be inserted into the inner side of the frame. The frame combining unit 303 may include a neck part 304 having a small width so that the frame combining unit 303 may be inserted into the inner side of the frame. A plurality of protrusions 305 (for example, protrusions 305a through 305g) may be provided on at least one surface of the neck part 304. That is, as can be seen from FIGS. 9 and 10, the neck part 304 has a smaller width relative to the width of the frame combining unit 303. The neck part 304 may also have a smaller length relative to the length of the frame combining unit 303. As shown in FIG. 10, the protrusions 305a through 305g may be disposed on an end surface of the neck part 304, which inserted into the inner side of the frame. In alternative embodiments, the plurality of protrusions may be disposed on more than one surface of the neck part. The type of a compression paddle 300 may be defined or determined depending on whether the plurality of protrusions 305 are accommodated in grooves of the compression paddle combining unit and depending on the number of protrusions which are accommodated. The example embodiment shown in FIGS. 9 and 10 is the same as the other example embodiments in that the compression paddle 300 includes a compression surface 301 that compresses the object 30 which may correspond to a breast and a compression surface frame 302 that is combined with the compression surface 301. Also, although not shown, the compression paddle combining unit may be provided in the same manner as that of the example embodiments of FIGS. 1 through 5.

FIG. 11 is a control block diagram of the X-ray imaging apparatus illustrated in FIG. 1.

In FIG. 11, solid lines refer to the flow of data. A detailed operation of the X-ray imaging apparatus will now be described with reference to FIG. 11.

As described above, if the compression paddle 100 is combined with the frame 70, the transmitting unit 80 determines whether the protrusions 105 are inserted into the grooves 72, and transmits signals to the controller 43 through the protrusions 105 of the frame combining unit 104. The type of the compression paddle 100 may vary according to the size of the object 30 which may correspond to a breast, and the controller 43 may detect the type of the compression paddle 100 and may determine a region in which the breast is put on the X-ray detection unit 60 (hereinafter, referred to as an object region). The region in which the breast is put on the X-ray detection unit 60 and which is determined by the type of the compression paddle 100 may be referred to as a first object region.

Additionally, a region in which the breast contacts the touch sensor 63 of the X-ray detection unit 60 and is put on the X-ray detection unit 60, may be defined or referred to as a second object region.

The collimator 42 of the X-ray generation unit 40 may be placed at the front of the X-ray source 41 and may adjust a radiation region of X-rays in which X-rays are radiated from the X-ray source 41. Thus, the controller 43 may control the collimator 42 so that the radiation region of X-rays generated in the X-ray source 41 may correspond to the object region. In detail, the controller 43 sets the radiation region of X-rays corresponding to the object region and transmits control signals that cause the X-rays generated in the X-ray source 41 to be radiated into the set radiation region, to the collimator 42. The controller 43 may control the collimator 42 based on signals regarding the type of the compression paddle 100 transmitted by the transmitting unit 80.

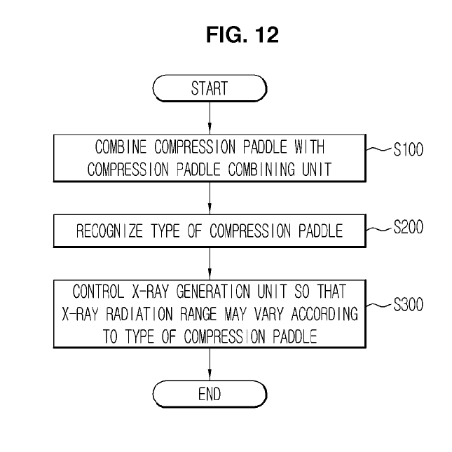

FIG. 12 is a flowchart illustrating a method of controlling an X-ray imaging apparatus, in accordance with an example embodiment.

Hereinafter, the method of controlling an X-ray imaging apparatus, in accordance with an example embodiment will be described.

Referring to FIG. 12, a compression paddle may be combined with a compression paddle combining unit of a frame (S100). For example, protrusions of a frame combining unit of the compression paddle may be combined with grooves of the compression paddle combining unit of the frame.

The method of controlling an X-ray imaging apparatus may further include combining the compression paddle with the frame in a first state in which the protrusions do not protrude, when the compression paddle is combined with the frame in regard to the combination of the compression paddle and converting the first state into a second state in which the protrusions protrude.

Also, an object (for example, a breast) may be put on an X-ray detection unit. For example, the compression paddle may be combined with the frame, and after the compression paddle is combined with the frame, the object may be placed on the X-ray detection unit.

Subsequently, the type of the compression paddle may be recognized using a plurality of protrusions provided at at least one surface of the compression paddle (S200). Recognizing the type of the compression paddle may be performed by recognizing whether the protrusions of the frame combining unit are combined with the grooves of the compression paddle combining unit. A transmitting unit placed at an inner side of the grooves may transmit data regarding the compression paddle through a controller. The controller may include one or more processors to perform the operations of the controller.

Subsequently, an X-ray generation unit may be controlled so that an X-ray radiation range may vary based on the type of the compression paddle (S300). Here, the controller may control a collimator of the X-ray generation unit and may adjust a radiation region of the X-rays. That is, the controller may determine a first object region according to an approximate size of the object by recognizing the type of the compression paddle and may primarily set the radiation region of the X-rays according to the first object region. In this way, the controller may control the collimator so that the X-rays generated in an X-ray source may be radiated into the set radiation region of the X-rays. Also, a second object region may be determined based on the object placed on an X-ray detection unit. In detail, the second object region may be determined according to the size and position of the object placed on a touch sensor. Thus, the radiation region of the X-rays may be secondarily set. For example, the radiation region of the X-rays may be set based upon, both the determined first object region and the determined second object region.

As described above, in an X-ray imaging apparatus and a method of controlling the same in accordance with the one or more of the example embodiments disclosed herein, since the type of a compression paddle may be physically recognized by protrusions of the compression paddle, there the possibility that an error will occur in recognizing the type of the compression paddle may be substantially decreased.

In addition, since the compression paddle may be combined with a frame by using the protrusions, the compression paddle may be stably combined with the frame.

The apparatuses and methods according to the above-described example embodiments may use one or more processors. For example, a processing device may be implemented using one or more general-purpose or special purpose computers, such as, for example, a processor, a controller and an arithmetic logic unit, a central processing unit (CPU), a graphics processing unit (GPU), a digital signal processor (DSP), a microcomputer, a field programmable array, a programmable logic unit, an application-specific integrated circuit (ASIC), a microprocessor or any other device capable of responding to and executing instructions in a defined manner.

The terms “module”, and “unit,” as used herein, may refer to, but are not limited to, a software or hardware component or device, such as a Field Programmable Gate Array (FPGA) or Application Specific Integrated Circuit (ASIC), which performs certain tasks. A module or unit may be configured to reside on an addressable storage medium and configured to execute on one or more processors. Thus, a module or unit may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules/units may be combined into fewer components and modules/units or further separated into additional components and modules.

Each block of the flowchart illustrations may represent a unit, module, segment, or portion of code, which comprises one or more executable instructions for implementing the specified logical function(s). It should also be noted that in some alternative implementations, the functions noted in the blocks may occur out of the order. For example, two blocks shown in succession may in fact be executed substantially concurrently or the blocks may sometimes be executed in the reverse order, depending upon the functionality involved.

The apparatuses and methods according to the above-described embodiments may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy disks, and magnetic tape; optical media such as CD ROM disks and DVDs; magneto-optical media such as optical discs; and hardware devices that are specially configured to store and perform program instructions, such as read-only memory (ROM), random access memory (RAM), flash memory, USB memory, and the like. Examples of program instructions include both machine code, such as produced by a compiler, and files containing higher level code that may be executed by the computer using an interpreter. The program instructions may be executed by one or more processors. The described hardware devices may be configured to act as one or more software modules in order to perform the operations of the above-described embodiments, or vice versa. In addition, a non-transitory computer-readable storage medium may be distributed among computer systems connected through a network and computer-readable codes or program instructions may be stored and executed in a decentralized manner. In addition, the computer-readable storage media may also be embodied in at least one application specific integrated circuit (ASIC) or Field Programmable Gate Array (FPGA).

Although example embodiments disclosed herein have been shown and described, it would be appreciated by those skilled in the art that changes may be made to these example embodiments without departing from the principles and spirit of the invention, the scope of which is defined in the claims and their equivalents.

What is claimed is:

1. An X-ray imaging apparatus comprising:
a frame;
an X-ray generation unit to generate and radiate X-rays toward an object;
an X-ray detection unit to detect X-rays transmitted by the object and to convert the detected X-rays into an electrical signal;
a compression paddle combinable with the frame and positioned adjacent to the X-ray detection unit to compress the object which is disposed between the compression paddle and X-ray detection unit;
a frame combining unit which forms a portion of the compression paddle and is combined with the frame; and
a plurality of protrusions disposed on at least one surface of the frame combining unit used to identify a type of the compression paddle.

2. The X-ray imaging apparatus of claim 1, further comprising a compression paddle combining unit disposed on the frame and adapted to be combined with the frame combining unit.

3. The X-ray imaging apparatus of claim 2, wherein the compression paddle combining unit comprises a plurality of grooves to accommodate the plurality of protrusions, wherein the compression paddle combining unit comprises a transmitting unit to generate signals to recognize insertion of the plurality of protrusions into the plurality of grooves.

4. The X-ray imaging apparatus of claim 1, wherein the compression paddle is adapted to move upward and downward relative to the frame.

5. The X-ray imaging apparatus of claim 2, wherein the frame combining unit is adapted to be inserted into an inner side of the frame and to contact the compression paddle combining unit, wherein the frame combining unit comprises a neck part inserted into the inner side of the frame, and the plurality of protrusions are provided on at least one surface of the neck part.

6. The X-ray imaging apparatus of claim 2, wherein the frame combining unit comprises an insertion part that protrudes from an upper portion and a lower portion of the frame combining unit, and
the compression paddle combining unit comprises an accommodation part having a shape corresponding to the insertion part.

7. The X-ray imaging apparatus of claim 3, wherein a surface on which the plurality of protrusions are disposed, corresponds to the surface of an inner side of the frame combining unit which connects to the compression paddle combining unit.

8. The X-ray imaging apparatus of claim 3, wherein the plurality of protrusions comprise a first state in which the protrusions protrude to be inserted into the grooves disposed in at least one surface of the compression paddle combining unit and a second state in which the protrusions are accommodated in the inner side of the frame combining unit.

9. The X-ray imaging apparatus of claim 1, further comprising a controller to identify the type of the compression paddle based on the plurality of protrusions and to control an X-ray radiation region of the X-ray generation unit, wherein the X-ray generation unit comprises an X-ray source to generate X-rays and a collimator to adjust a radiation region of the X-rays radiated from the X-ray source, and
the controller controls the X-ray radiation region of the collimator according to the type of the compression paddle.

10. A method of controlling an X-ray imaging apparatus, the method comprising:
combining a compression paddle with a frame of the X-ray imaging apparatus;
recognizing a type of the compression paddle based on a plurality of protrusions disposed on at least one surface of the compression paddle; and
controlling an X-ray generation unit to generate X-rays based on the recognized type of the compression paddle.

11. The method of claim 10, wherein the recognizing comprises determining whether the protrusions of the frame combining unit are inserted into grooves of a compression paddle combining unit which is combined with the frame, further comprising transmitting data regarding the compression paddle to a controller which performs the controlling of the X-ray generation unit.

12. The method of claim 10, wherein the combining of the compression paddle comprises combining the compression paddle with the frame so that the plurality of protrusions protrude into grooves of a compression paddle combining unit which forms a portion of the frame.

13. The method of claim 10, further comprising placing an object on an X-ray detection unit, the object being placed between the compression paddle and the X-ray detection unit, and subsequently combining the compression paddle with the frame.

14. The method of claim 10, further comprising placing an object on an X-ray detection unit, the object being placed between the compression paddle and the X-ray detection unit, after combining the compression paddle with the frame.

15. The method of claim 10, wherein the controlling of the X-ray generation unit comprises:
determining a first object region according to the recognized type of the compression paddle; and
determining a second object region according to an object placed on a X-ray detection unit, the object being placed between the compression paddle and the X-ray detection unit.

16. The method of claim 10, wherein the controlling of the X-ray generation unit comprises controlling a collimator and adjusting a radiation region of the X-rays, wherein the controlling of the collimator comprises determining an object region according to a size of the object by recognizing the type of the compression paddle combined with the frame and setting a radiation region of the X-rays to correspond to the object region, wherein the controlling of the collimator further comprises controlling the collimator so that the X-rays generated by the X-ray generation unit are radiated into the set radiation region.

17. An X-ray imaging apparatus comprising:
a frame;
a compression paddle to compress an object disposed between the compression paddle and an X-ray detection unit;
a frame combining unit connected to the compression paddle, which includes a plurality of protrusions disposed on at least one surface of the frame combining unit; and
a compression paddle combining unit connected to the frame and to the frame combining unit, which includes a plurality of grooves disposed on at least one surface of the compression paddle combining unit to accommodate the plurality of protrusions.

18. The X-ray imaging apparatus of claim 17, further comprising a controller to recognize a type of the compression paddle based upon a determination as to a number of protrusions which are inserted into the plurality of grooves, wherein the compression paddle combining unit further includes a transmitting unit to transmit a signal to the controller indicating whether a first protrusion of the frame combining unit is inserted into a first groove of the compression paddle combining unit.

19. The X-ray imaging apparatus of claim 17, wherein the transmitting unit includes:
a transmitting body;
a connection part disposed on the transmitting body;
a first terminal to receive a force when the first protrusion is inserted into the first groove; and
a metal plate connected to the first terminal on a first end and connected to the transmitting body on a second end, to contact the connection part when the first terminal receives the force,
wherein the transmitting unit transmits the signal to the controller when the connection part is contacted by the metal plate.

20. The X-ray imaging apparatus of claim 17, wherein the compression paddle includes:
a compression surface to compress the object; and
a first compression surface frame and a second compression surface frame adapted to accommodate the compression surface,
wherein the frame combining unit is integrally formed together with the first compression surface frame and the second compression surface frame.
